(19) United States
(12) Patent Application Publication
(10) Pub. No.: US 2011/0132995 A1
(43) Pub. Date: Jun. 9, 2011

(54) FRAGRANCE DISPENSER WITH REPLACEABLE CARTRIDGES

(75) Inventor: Klas Fredrik Perman, Raleigh, NC (US)

(73) Assignee: USB FLOWER, LTD, Limassol (CY)

(21) Appl. No.: 12/963,274

(22) Filed: Dec. 8, 2010

Related U.S. Application Data
(60) Provisional application No. 61/285,015, filed on Dec. 9, 2009.

(51) Int. Cl. A61L 9/02

(52) U.S. Cl. 239/34

(57) ABSTRACT

Embodiments of the present invention provide a fragrance dispenser, comprising a housing, wherein the housing comprises a compartment for holding a replaceable scent cartridge and wherein the compartment comprises a removable cover. The fragrance dispenser further comprises a heating element, and a mechanism for removably connecting the dispenser to a power source. In at least some embodiments, the housing is an ornamental shape. In at least some embodiments, the replaceable scent cartridge comprises a pad to which fragrance oil has been applied.

### CROSS REFERENCE TO RELATED APPLICATIONS

The present application claims the benefit of U.S. Provisional Patent Application No. 61/285,015 filed Dec. 9, 2009, filed in the name of Klas Fredrik Perman and entitled “Fragrance Dispenser with Replacement Cartridges”, the contents of which is incorporated by reference herein in its entirety.

### BACKGROUND

Fragrance dispensers are enjoyed by many people who like to have a pleasant odor wafting throughout their surroundings. Many currently available scent dispensers must be plugged into a wall electrical outlet, which limits where they can be located. Further, the scent disappears as the source of the scent is used up. Many scent dispensers must simply be disposed of when there is no longer a pleasant fragrance. Finally, many scent dispensers are not particularly attractive to look at, which serves as yet a further limitation as to where the user would like for the dispenser to be located while it is being used.

### SUMMARY OF THE INVENTION

Embodiments of the present invention provide a fragrance dispenser, comprising a housing, wherein the housing comprises a compartment for holding a replaceable scent cartridge and wherein the compartment comprises a removable cover. The fragrance dispenser further comprises a heating element, and a mechanism for removably connecting the dispenser to a power source. In at least some embodiments, the housing is an ornamental shape. In at least some embodiments, the replaceable scent cartridge comprises a pad to which fragrance oil has been applied.

### BRIEF DESCRIPTION OF THE DRAWINGS

FIG. 1 depicts a view of an exemplary embodiment of the present invention wherein the fragrance dispenser is fully assembled.

FIG. 2 depicts a view of an exemplary embodiment of the present invention wherein the removable cover has been removed and the replaceable scent cartridge has been taken out of the compartment.

FIG. 3 depicts an exploded view of an exemplary embodiment of the present invention.

FIG. 4 depicts a view of exemplary embodiments of the present invention.

### DETAILED DESCRIPTION

The present invention will now be described in terms of specific, example embodiments. It is to be understood that the invention is not limited to the example embodiments disclosed. It should also be understood that not every feature of the methods described is necessary to implement the invention as claimed in any particular one of the appended claims. Various elements and features of various embodiments are described in order to fully enable the invention.

Embodiments of the present invention provide a fragrance dispenser (also referred to herein as a dispenser) for wafting a fragrance into the surrounding environment, the fragrance dispenser comprising a housing, wherein the housing comprises a compartment for holding a replaceable scent cartridge and a heating element. The dispenser further comprises a mechanism for removably connecting the dispenser to a power source.

The power source can be any source that provides an electrical current for heating up the heating element in the fragrance dispenser, thereby enhancing release of the fragrance. In some embodiments, the mechanism for removably connecting the dispenser to a power source is a USB connection (or plug). The USB connection allows the user to activate the fragrance dispenser by plugging it into, for example, a USB port of a computer, thereby allowing the user to enjoy the scent while using the computer.

In some embodiments, the mechanism for removably connecting the dispenser to a power source is a plug or adaptor for an automobile cigarette lighter socket. The plug for the cigarette lighter socket allows the user to enjoy the scent while traveling in an automobile. In some embodiments, the fragrance dispenser may have interchangeable mechanisms for removably connecting the dispenser to a power source. For example, a user could attach a USB connection to the fragrance dispenser for one use and a cigarette lighter plug for another use of the same fragrance dispenser. One of skill in the art could envision other mechanisms for removably connecting the dispenser to a power source, and such mechanisms would fall into the scope of the presently claimed invention. An example of another such mechanism is a connector for operably attaching the dispenser to a portable battery pack.

In at least some embodiments, the housing is an ornamental shape. The ornamental shape may be any shape pleasing to the user. Ornamental shapes may include shapes such as flowers, trees, animals, toys, vehicles, or foods. Other ornamental shapes may include shapes representing holiday themes, such as, but not limited, to jack-o-lanterns, Christmas trees, gingerbread men, birthday cakes, turkeys, horns of plenty, heart shapes, or roses. One of skill in the art would recognize that the precise shape of the fragrance dispenser may differ but will still fall within the scope of the present invention. In some embodiments, the scent may be selected to correlate, or be associated with, the ornamental shape. For example, the rose-shaped dispenser may be provided with a rose scented cartridge, the Christmas tree-shape dispenser may be provided with a pine-tree scented cartridge, or the gingerbread man-shaped dispenser may be provided with a gingerbread scented cartridge.

In at least some embodiments, the replaceable scent cartridge comprises a pad to which fragrance oil has been applied. In some embodiments, the replaceable scent cartridge comprises a pad within a solid holder. The holder may be constructed of any material that is suitable for holding the pad and the scented oil, and that can withstand the heating of the fragrance dispenser. Examples of suitable materials include glass and metal. In one exemplary embodiment, the solid holder is aluminum.

In at least some embodiments, the replaceable scent cartridges may be purchased separately from the dispenser, so that a user can obtain refill cartridges for replacing a scent cartridge when it is spent. The replaceable scent cartridges prevent the user from having to handle fragrance oil liquids, as the entire cartridge is replaced when the scent wanes and the user wishes to refresh it. This is desirable, as there is always a risk when handling liquids near electronics that the fragrance oils might get onto or into the power source (such as a computer) and harm the electronic workings of the power source.

The replaceable scent cartridges maintain a maximum shelf life if they are protected from exposure to the air. Exposure to air may cause the oils in the pad to dry up, thereby reducing the effectiveness of the fragrance dispenser. Thus, the dispenser and the refill cartridges are packaged in a way that protects the cartridge from the drying effects of the air. In at least one embodiment, the dispenser and/or the replaceable cartridges are sealed in foil packets prior to use.

The compartment for holding the replaceable scent cartridge comprises a removable cover. The removable cover may be attached to the housing by any suitable means. In one exemplary embodiment, the cover is a snap-on cover. In at least some embodiments, the cover includes a plurality of openings through which the scent from the scent cartridge is released.

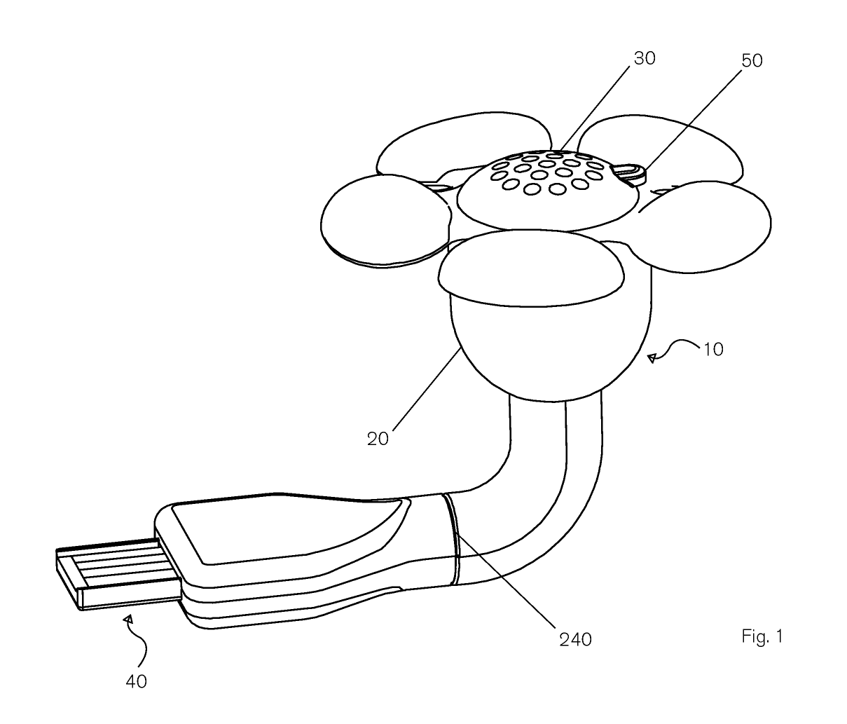

With reference to the figures, the following description is based on an exemplary implementation of an embodiment of the invention. Reference numbers are used consistently throughout FIGS. 1-4 herein.

In FIG. 1, an embodiment of the fragrance dispenser is depicted as it looks when fully assembled. In the exemplary embodiment shown in FIG. 1, the ornamental shape of the housing 10 is a flower shape. The housing 10 of the fragrance dispenser comprises a compartment 20 that holds a replaceable scent cartridge (not visible) and is covered by a removable cover 30. The fragrance dispenser is plugged into a power source by the mechanism for removably connecting the fragrance dispenser to a power source, which in the embodiment of FIG. 1 is a USB plug. The removable cover 30 can be snapped off by pulling up on the tab 50.

The housing 10 of the fragrance dispenser may be made of any suitable material, such as plastic, glass or metal. The material must be capable of holding the replaceable scent cartridge and must be able to withstand the heat generated by the heating element when the fragrance dispenser is connected to a power source. In the embodiment shown in FIG. 1, the ornamental shape of the housing 10 comprises the petals of the flower and the flower stem. The removable cover 30 may be made of the same or a different material as the housing 10. In at least some embodiments, such as the one depicted in FIG. 1, the removable cover 30 includes a plurality of openings through which the scent from the replaceable scent cartridge is released. In at least some embodiments, the part of the housing that makes up the “stem” reaching from the mechanism for removably connecting the dispenser to a power source is rotatably adjustable. That is, the seam 240 is constructed so that the housing 20, or the head of the flower in FIG. 1, can be rotated relative to the USB plug, thus changing the direction in which the scent is released.

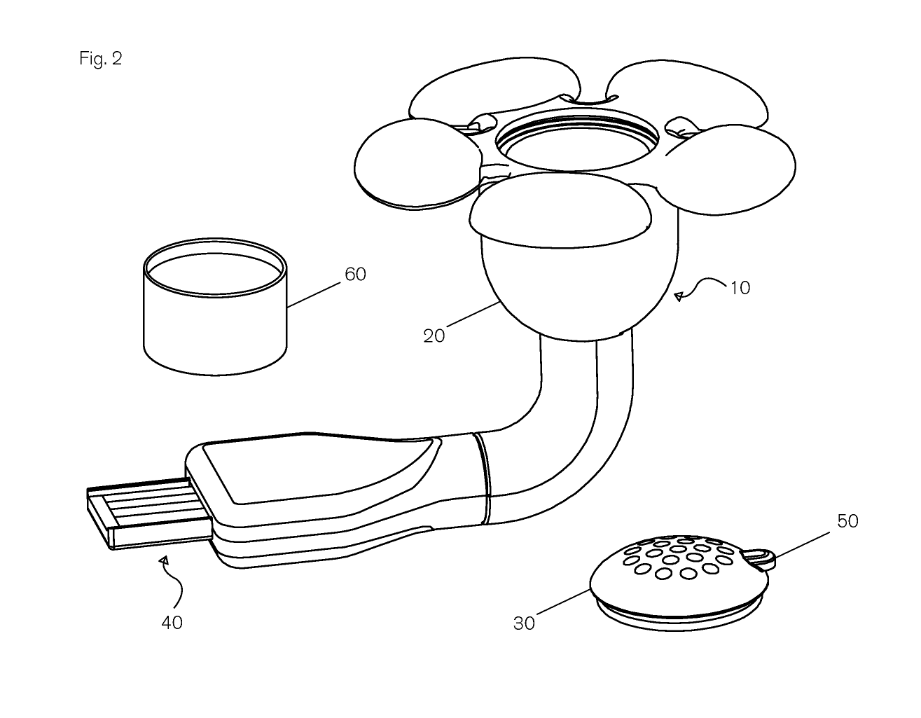

FIG. 2 depicts the same view of an embodiment of the fragrance dispenser as is shown in FIG. 1, except that in FIG. 2 the removable cover 30 is removed from the compartment 20 of the housing 10 and the replaceable scent cartridge 60 is removed as well. The replaceable scent cartridge 60 of the fragrance dispenser may be made of any suitable material, such as plastic, glass or metal.

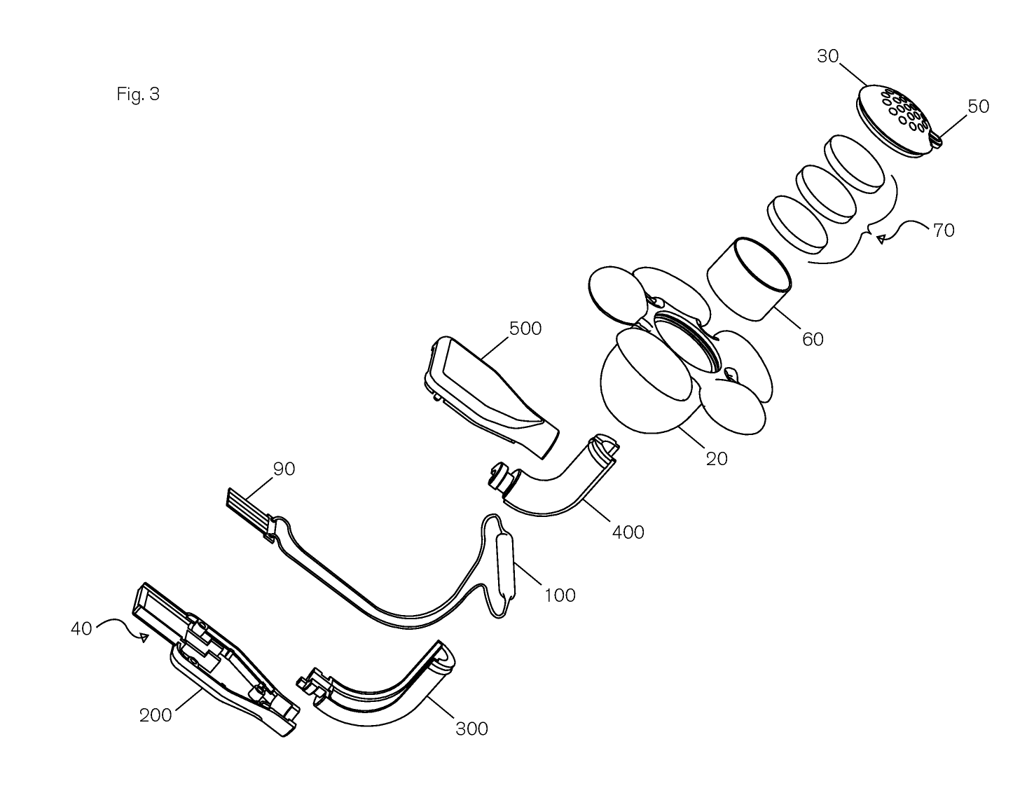

In FIG. 3, an exploded view of an embodiment of the fragrance dispenser is depicted. The housing 10 comprises the compartment 20 for holding the replaceable scent cartridge 60. The replaceable scent cartridge 60 comprises at least one fragrance pad 70 to which fragrance oil has been added. In the embodiment shown in FIG. 3, three fragrance pads 70 are contained in the cartridge 50. The number and thickness of the fragrance pads 70 may differ and may be dictated at least in part by the manufacturing process. It is understood that the number or thickness of the fragrance pads 70 may differ and still fall within the scope of the present invention.

The fragrance pads may be made of any suitable absorbent material, such as a paper or paper pulp product, a cotton product, or any other material that can absorb and retain fragrance oil.

FIG. 3 further shows the mechanism for connecting to a power source 40 disassembled to show the outer shell 40 and the electrical components of the plug attached to a heating element 100. In the embodiment shown in FIG. 3, the wires to the heating element are housed within the flower stem, inside components 200, 300, 400, and 500. Components 200 and 500 fit together around the wires of the heating element mechanism 100, leaving the plug 90 accessible to be plugged into the power source. Components 300 and 400 fit together further up the stem of the flower, above components 200 and 500, and closer to where the heating element is situated. The heating element 100 rests inside the compartment 20 of the housing 10, just below the replaceable scent cartridge 60.

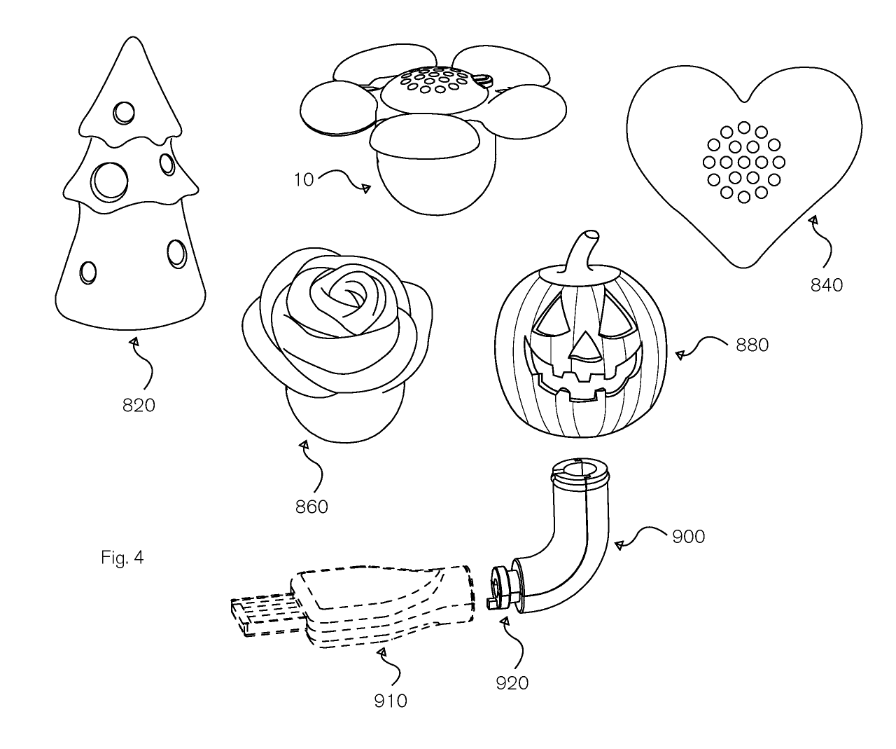

FIG. 4 depicts alternative embodiments of the fragrance dispenser. The ornamental shapes of the housing of embodiments of the present invention may include, but are not limited to, pine tree or Christmas tree shapes 820, flower shapes 10, heart shapes 840, rose shapes 860 and jack-o-lantern or pumpkin shapes 880. One of skill in the art understands that any ornamental shape could be used with the present invention and would still fall within the scope of the invention.

FIG. 4 further depicts a mechanism for removably connecting the fragrance dispenser to a power source. As has already been noted above, in some embodiments the mechanism for removably connecting the fragrance dispenser to a power source is a USB plug 910. The USB plug is useful, for example, for connecting the fragrance dispenser to a USB port of a desktop or laptop computer. In some embodiments, the mechanism for removably connecting the fragrance dispenser to a power source is a plug for connecting to a cigarette lighter socket in an automobile. In some embodiments, the mechanism for removably connecting the fragrance dispenser to a power source is a connector for connecting the fragrance dispenser to a portable battery pack, thus allowing the user to carry and use the fragrance dispenser where desired.

Note that the section of the fragrance dispenser that connects to the mechanism for removably connecting the fragrance dispenser to a power source 900 is a combination of components 300 and 400 shown in FIG. 3. In at least some embodiments, element 900 is configured so that the type of connector, or plug (such as USB plug 910 in the example shown in FIG. 4), is exchangeable at the connector site 920. This feature allows the user to change the type of power source to which the fragrance dispenser can be connected, thus increasing the versatility of the fragrance dispenser.

While certain exemplary embodiments have been described and shown in the accompanying drawings, it is to be understood that such embodiments are merely illustrative of and not restrictive on the broad invention, and that this invention not be limited to the specific constructions and arrangements shown and described, since various other changes, combinations, omissions, modifications and substitutions, in addition to those set forth in the above paragraphs, are possible. Those skilled in the art will appreciate that various adaptations and modifications of the just described embodiments can be configured without departing from the scope and spirit of the invention. Therefore, it is to be understood that, within the scope of the appended claims, the invention may be practiced other than as specifically described herein.

What is claimed is:

1. A fragrance dispenser comprising:
a. a housing comprising:
i. a compartment for holding a replaceable scent cartridge, wherein the compartment comprises a removable cover;
ii. a heating element; and
b. a mechanism for removably connecting the fragrance dispenser to a power source.

2. The fragrance dispenser of claim 1, wherein the housing comprises an ornamental shape.

3. The fragrance dispenser of claim 1, wherein the replaceable scent cartridge comprises a pad to which fragrance oil has been applied.

4. The fragrance dispenser of claim 1, wherein the mechanism for removably connecting the dispenser to a power source is a USB attachment.

5. The fragrance dispenser of claim 1, wherein the mechanism for removably connecting the dispenser to a power source is an adaptor for plugging into a vehicle cigarette lighter socket.

6. The fragrance dispenser of claim 2, wherein the ornamental shape is selected from the group consisting of a pine tree or Christmas tree shape, a flower shape, a heart shape, a rose shape, and a jack-o-lantern or pumpkin shape.

7. The fragrance dispenser of claim 1, wherein the removable cover includes a plurality of openings through which the scent from the scent cartridge is released.

8. The fragrance dispenser of claim 1, wherein the housing is rotatably adjustable so that the direction in which the removable cover is pointing is changeable.

9. The fragrance dispenser of claim 1, wherein the mechanism for removably connecting the dispenser to a power source is an adaptor for plugging into a portable battery pack.
